(19) United States
(12) Patent Application Publication
(10) Pub. No.: US 2018/0042827 A1
(43) Pub. Date: Feb. 15, 2018

(54) METHOD OF PROTECTING SKIN FROM ULTRAVIOLET RADIATION

(71) Applicant: FIC BIOTECH INC., Taipei City (TW)

(72) Inventors: Tsan-Zon LIU, Taipei City (TW); Chun-Hsien CHEN, Taipei City (TW)

(21) Appl. No.: 15/621,839

(22) Filed: Jun. 13, 2017

(30) Foreign Application Priority Data
Aug. 12, 2016 (TW) 105125694

(51) Int. Cl. A61K 8/37; A61Q 17/04

(52) U.S. Cl. CPC A61K 8/37; A61Q 17/04

(57) ABSTRACT

Disclosed is method of protecting skin from ultraviolet radiation which comprises the step of administering a skin protective composition. The skin protective composition disclosed in the present invention includes a compound, which not only can protect normal human skin against UV-induced damage, but also exhibits protective benefits on the skin of patients with G6PD-deficiency.

### CROSS REFERENCE TO RELATED APPLICATION

This application claims the benefits of the Taiwan Patent Application Serial Number 105125694, filed on Aug. 12, 2016, the subject matter of which is incorporated herein by reference.

### BACKGROUND OF THE INVENTION

### 1. Field of the Invention

The present invention relates to a method of protecting skin from ultraviolet radiation and, more particularly, to a method of protecting skin from ultraviolet radiation by administering a skin protective composition.

### 2. Description of Related Art

Ultraviolet radiation in sunlight is considered as the main culprit that damage the skin, wherein ultraviolet A (UVA) has the strongest penetration through the skin among three types of ultraviolet, and the content is also the most of the three types of ultraviolet (about 96%). Thus, UVA can cause severe damages to the skin. Particularly, UVA can cause even more severe damages to the patients with glucose-6-phosphate dehydrogenase (G6PD) deficiency than normal individual. Therefore, it becomes an important issue to protect skin from damages caused by ultraviolet irradiation.

G6PD deficiency is one of the most common enzymopathy affecting more than 400 million people worldwide. The lack of enzyme activity predisposes G6PD-deficient individual to red blood cell (RBC)-related disorders such as favism as well as non-RBC-related disorders, such as diabetes and hypertension [Yang H C et al. Free radical research. 2016; 50(10):1047-64].

The severe reduction or absent of G6PD in G6PD-deficient patients was ascribable to point mutation leading to amino acid substitution and diminished enzyme activity in their cells including fibroblasts. G6PD catalyzes the conversion of glucose-6-phosphate (G6P) to 6-phosphogluconate with concomitant generation of NADPH [Hecker P A et al. American journal of physiology Heart and circulatory physiology. 2013; 304(4):H491-500]. The latter compound functions as a major reductive equivalent for the regeneration of oxidized GSSG back to GSH catalyzed by GSH reductase [Ye J et al. Annals of clinical and laboratory science. 2000; 30(1):65-71, Hsieh T J et al. Food and chemical toxicology. 2004; 42(5):843-50]. In addition, NADPH also serves as a cofactor in the synthesis of nitric oxide (NO)[Forstermann U et al. European heart journal. 2012; 33(7):829-37]. Meanwhile, the reduced GSH can be used to remove reactive oxygen species (ROS), such as H₂O₂ to alleviate the oxidative damages caused by ROS. Thus, it can be anticipated that G6PD-deficient patients will be prone to enhanced susceptibility to oxidative damages caused by the exposure of UV irradiation. In Taiwan, majority of patients suffering from G6PD deficiency are HaKKa people and their skins are much more vulnerable to UV-mediated oxidative damages as compared to those of the non-HaKKa population because most of them work as farmers [Hu R et al. Int J Clin Exp Pathol. 2015; 8(11):15013-15018]. For this reason, it is desirable to develop a prophylactic product for skin that can protect not only normal individual, but also G6PD-deficient patients from oxidative damages caused by UV irradiation.

Based on this premise, it is desirable to develop a product that can protect not only normal people, but also G6PD deficiency patients from damages caused by UV irradiation.

### SUMMARY OF THE INVENTION

The objective of the present invention is to provide a prophylactic skin protective composition, which not only protects the skin of normal person from ultraviolet radiation damage but also effectively protect the skin of glucose-6-phosphate dehydrogenase (G6PD) deficient patients. Therefore, the skin damages of G6PD-deficient patients caused by ultraviolet radiation can be alleviated.

To achieve this objective, the present invention provides a method of protecting skin of G6PD deficient patients from ultraviolet radiation, which comprises the step of administering a skin protective composition including an effective amount of a compound (I), which has been shown to be membrane permeable:

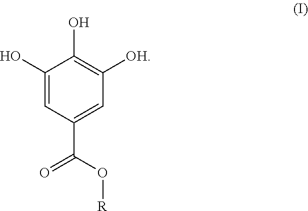

Herein, R represents unsubstituted C₁₋₆ alkyl group and is preferably methyl group. In addition, the source of compound (I) is not particularly limited which can be purchased on the market or can be synthesized naturally or artificially. Our experimental evidence suggests that compound (I) possesses multiple functions including effective scavenging of reactive oxygen species (ROS), reducing lipid peroxidation, preventing intracellular glutathione (GSH) depletion, mitochondrial calcium overload, and collagen breakdown. All these effects instigated by compound (I) can thus effectively protect human fibroblasts from apoptotic lethality caused by exposure to UV irradiation. Hence, compound (I) may protect the skin from UV damages and reduce the damages caused by UVA.

In the present invention, the skin protective composition may further comprise a pharmaceutically or physiologically acceptable carrier, diluent, or excipient, and the application method or type is not particularly limited. For example, the skin protective composition may be formulated into a skin topical composition, an oral composition, an injection composition, and a nasal inhalation composition, and can be applied as lotions, creams, ointments, gels, oil, soap, sprays, drinking water, tablets, capsules, granules, powders, inhalants, injections.

In the present invention, compound (I) may be used with combination of other active components as needed to enhance the skin anti-radiation or other effects. For example, when the skin protective composition is formulated into a skin topical composition, the skin protective composition may further comprise other components such as whitening agent, moisturizers, UV absorbers, skin nutrients so that the desired synergistic effects can be achieved when applying an effective amount of the skin protective composition.

Accordingly, compound (I) of the present invention used as the active component of the skin protective composition may efficiently improve the tolerance to UV radiation, slow down the aging and wrinkle caused by UV radiation. In this invention, we have evidence to substantiate that despite a drastic difference in apoptotic cell population (as reflected by the percentage of TUNEL-positive cells) when both normal and G6PD-deficient fibroblasts are exposed to UV-A irradiation (50 KJ/cm²), we have found that compound (I) can effectively protect G6PD-deficient fibroblasts from UV-A-instigated apoptotic lethality even more efficiently than normal fibroblasts. Based on this finding, we suggest that compound (I) can be an excellent prophylactic skin protection agent for G6PD-deficient patients.

In summary, the present invention relates to the method of protecting skin from ultraviolet radiation which comprises the step of administering a skin protective composition containing compound (I-1) as an effective ingredient observed using fibroblasts of human origin (normal HFF-3 and G6PD-deficient HFF-1 cells) as the experimental cell models. More particularly, the present invention is an excellent protective agent for the skin of G6PD-deficient patients whose fibroblasts are highly vulnerable to UVA-provoked oxidative damages owing to the lack of the production of NADPH necessary for the regeneration of oxidized GSSG back to GSH as well as the production of nitric oxide (NO). Thus, topical application of the present invented composition will allow G6PD-deficient fibroblasts to be protected by overcoming the enhanced sensitivity against UVA-provoked apoptotic lethality, so that the biosynthesis of collagen and its breakdown will be effectively maintained and inhibited. Taken together, the present invention can also be an effective anti-wrinkling composition specifically more useful for G6PD-deficient patients.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a fluorescence image of intracellular ROS detection of HFF3 cells of one embodiment of the present invention.

FIG. 2 is a fluorescence image of intracellular ROS detection of HFF1 cells of one embodiment of the present invention.

FIG. 3 is a fluorescence image of intracellular lipid peroxidation detection of HFF3 cells of one embodiment of the present invention.

FIG. 4 is a fluorescence image of intracellular lipid peroxidation detection of HFF1 cells of one embodiment of the present invention.

FIG. 5 is a fluorescence image of intracellular GSH depletion detection of untreated HFF3 cells and HFF1 cells of one embodiment of the present invention.

FIG. 6 is a fluorescence image of intracellular GSH depletion detection of HFF3 cells and HFF1 cells treated with compound (I) of one embodiment of the present invention.

FIG. 7 is a fluorescence image of cytoplasmic and mitochondrial Ca²⁺ detection of HFF3 cells.

FIG. 8 is a fluorescence image of cytoplasmic and mitochondrial Ca²⁺ detection of HFF1 cells.

FIG. 9 is a fluorescence image of TUNEL analysis of HFF3 cells of one embodiment of the present invention.

FIG. 10 is a quantitative analysis diagram of TUNEL analysis of HFF3 cells of one embodiment of the present invention.

FIG. 11 is a fluorescence image of TUNEL analysis of HFF1 cells of one embodiment of the present invention.

FIG. 12 is a quantitative analysis diagram of TUNEL analysis of HFF1 cells of one embodiment of the present invention.

### DETAILED DESCRIPTION OF THE PREFERRED EMBODIMENT

Compound (I) of the present invention may be commercially available, extracted from natural products, or artificial synthesized. For example, compound (I) may be synthesized through the following esterification:

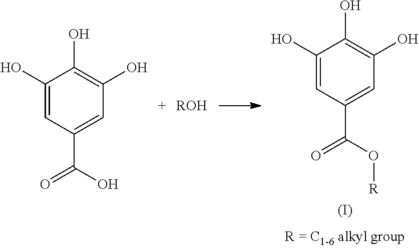

Compound (I-1) will be used to discuss the effects on mitigation UV radiation in the following descriptions.

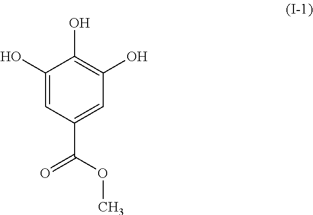

When carboxylic group of gallic acid is substituted by a methyl group, compound (I-1) (referred to as methyl gallate) becomes non-polar in nature and thus capable of penetrating into the cells to act as an excellent antioxidant.

[Experimental Materials]

[Cell Line]

Human foreskin fibroblast (HFF-3) and G6PD deficient skin fibroblast (HFF-1, G6PD^{1379T} type) are established according to Ho H Y, et al. [Free radical biology & medicine. 2000; 29(2):156-69.] with enhanced oxidative stress and accelerated cellular senescence in glucose-6-phosphate dehydrogenase (G6PD)-deficient human fibroblasts.

[Cell Culture Medium]

The culture medium comprises Dulbecco's Modified Eagle's Medium (GIBCO® DMEM), GIBCO® Dulbecco's Phosphate-Buffered Saline (DPBS), Fetal bovine serum (FBS), and Gibco® Trypsin-EDTA (lx).

[Compound (I-1) Solution]

Compound (I-1) solution is prepared by dissolving compound (I-1) in dimethyl sulfoxide (DMSO).

[Cell Culture]

HFF3 and HFF1 cells were cultured in 1×DMEM medium containing high glucose (4.5 g/L), 5% FBS, and 1% penicillin/streptomycin and incubated at 37° C.

[Addition of Compound (I-1) and UVA Irradiation]

When cells were seeded in culture dish for overnight incubation, 200 μM of compound (I-1) was added to the cells for 2 hours before UVA irradiation.

[Fluorescent Dyes Used]

Reactive oxygen species (ROS) fluorescent dye: when 10 μM of 2′,7′-dichlorodihydrofluorescindiacetate (DCF-DA) enters the cells, DCF trapped is oxidized by the ROS and emits fluorescent light (excitation light: 488 nm, scattered light: 525 nm).

Lipid peroxidation fluorescent dye: 10 μM of 4,4-difluoro-4-bora-3a,4a-diaza-s-indacene (C₁₁-BODIPY^{581/591}) is a fluorescent probe possessing a double bond structure mimicked to a lipid and can be inserted on the cell membrane. When the ROS attacks the C₁₁-BODIPY^{581/591} on the cell membrane, it will emit fluorescent light (excitation light: 488 nm, scattered light: 510-665 nm).

Glutathione (GSH) fluorescent dye: 10 μM of 5-chloromethyl fluorescein diacetate (CMF-DA). When CMF-DA enters the cells, the trapped CMF reacts with the GSH to form a fluorescent compound which to emit fluorescent light (excitation light: 492 nm, scattered light: 517 nm).

Calcium mobilization detection dye: 1.5 μM of RHOD-2/AM (rhod-2) is a cell-permeable fluorescent probe for measuring calcium (Ca²⁺) in mitochondria (excitation light: 543 nm, scattered light: 581 nm), and 2 μM of FLUO-4/AM (fluo-4) is a cell-permeable fluorescent probe for measuring calcium (Ca²⁺) in cytoplasm (excitation light: 488 nm, scattered light: 516 nm).

[Intracellular ROS Detection]

HFF-3 and HFF-1 cells were cultivated and irradiated with various dosages of UVA (25, 50 and 100 KJ/cm²). The culture medium was then replaced with new medium and treated with 10 μM of DCF-DA probe for 30 minutes at 37° C. in the dark. Cells were detached by trypsinization, collected by centrifugation and resuspended in PBS. The intracellular ROS, as reflected by the DCF green fluorescence, were then observed using laser scanning confocal microscope.

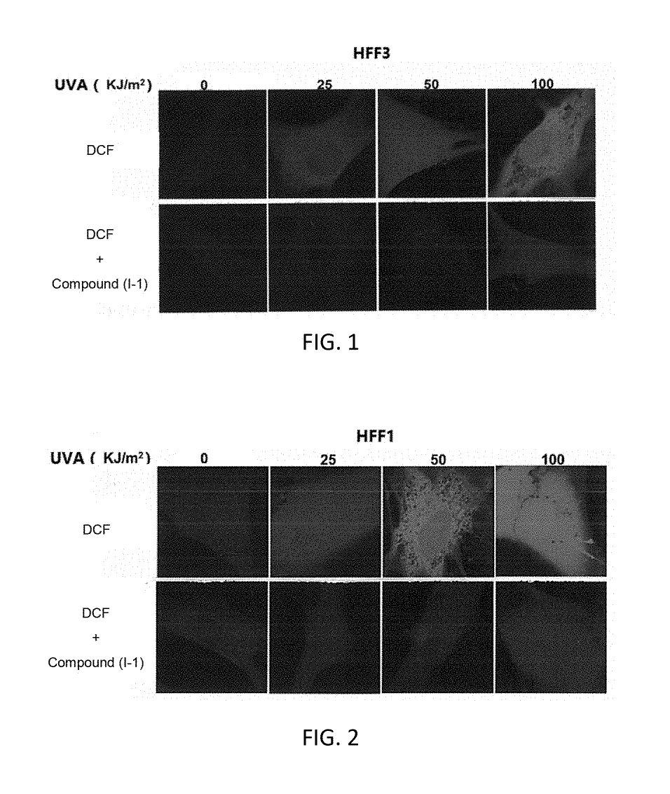

As indicated in FIG. 1 and FIG. 2, production of intracellular ROS triggered by UVA irradiation, as reflected by the intensity of DCF fluorescence, increase progressively in a dose-dependent manner. However, ROS generated by UVA-treated HFF-1 cells was found to be much more abundant than HFF-3 cells indicating that former cell type was highly oxidative-stressed. Remarkably, when both HFF-3 and HFF-1 cells were treated with compound (I-1) (200 μM) prior to UVA irradiation, the production of ROS in both cell type was almost completely suppressed (refer to the bottom rows of FIG. 1 and FIG. 2). This finding suggests that compound (I-1) is an excellent ROS scavenger as well as UVA protector.

[Intracellular Lipid Peroxidation]

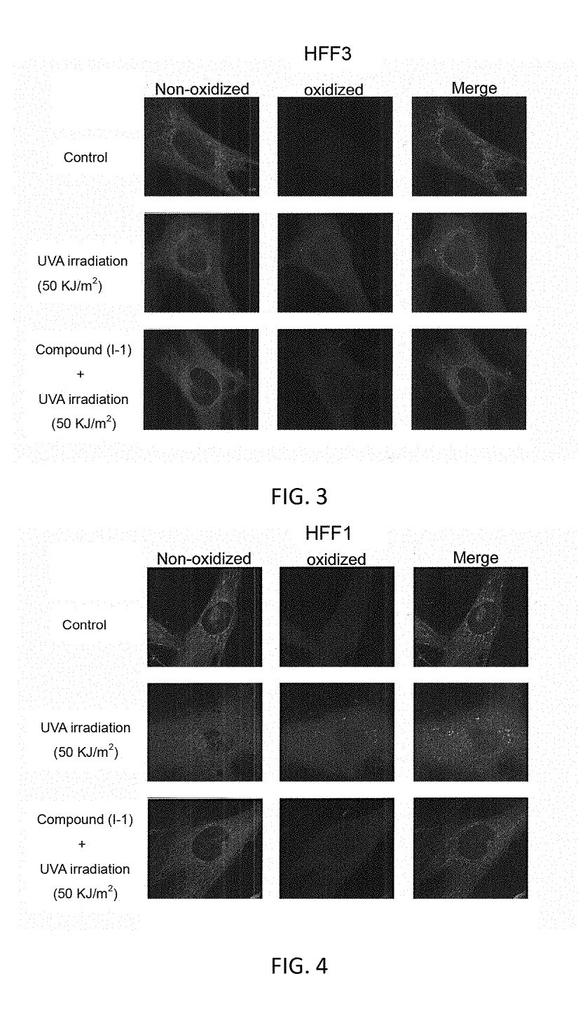

After treating 10 μM C₁₁-BODIPY^{581/591} for 30 minutes at 37° C., the cells were washed with HEPES buffer (4-(2-hydroxyerhyl)-piperazine-1-erhanesulfonic acid, HEPES) and were observed by using the laser scanning confocal microscope. The microscope images were shown in FIG. 3 and FIG. 4.

The fluorescent color changes from red to green, when cellular C₁₁-BODIPY^{581/591} is oxidized by ROS. Therefore, by comparing the fluorescent images of upper two rows of FIG. 3 and FIG. 4, green fluorescent intensity of HFF3 and HFF1 irradiated by 50 KJ/m² UVA were stronger than the non-irradiated control groups. This indicates that UVA irradiation causes intracellular lipid peroxidation. Furthermore, according to the fluorescent images of HFF3 and HFF1 cells which were treated with compound (I-1), the green fluorescent intensity of compound (I-1) treated cells were weaker than that of the only UVA-treated cells (refer to the bottom row of FIG. 3 and FIG. 4). This implies that compound (I-1) can reduce the damages of UVA irradiation-triggered lipid peroxidation in HFF3 and HFF1 cells.

[Intracellular Glutathione (GSH) Depletion]

HFF-3 and HFF-1 cells were cultivated and irradiated with various dosages of UVA (25, 50 and 100 KJ/cm²). The culture medium was then replaced with new medium and treated with 10M CMF-DA probe for 30 minutes at 37° C. in the dark. Cells were detached by trypsinization, collected by centrifugation and resuspended in PBS. The intracellular GSH contents, as reflected by the green fluorescence intensity of CMF-GSH, were then observed using laser scanning confocal microscope.

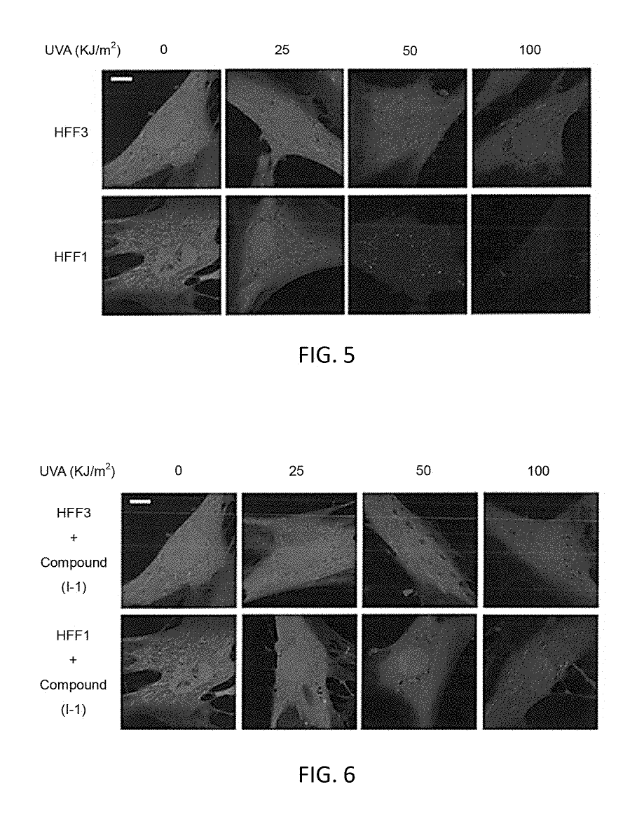

As indicated in FIG. 5, after both HFF-3 and HFF-1 cells receiving various dosages of UVA irradiation, the green fluorescence images of both cell type decrease progressively in a dose-dependent manner indicating that intracellular GSH depletion phenomenon has occurred. However, the extent of GSH depletion caused by UVA-treated HFF-1 cells was found to be more severe than HFF-3 cells. Remarkably, when both HFF-3 and HFF-1 cells were treated with compound (I-1) (200 μM) prior to UVA irradiation, a nearly complete protection of GSH depletion can be observed (FIG. 6).

[Calcium Detection of Cytoplasm and Mitochondria]

HFF-3 and HFF-1 cells were cultivated and irradiated with UVA (50KJ/cm²). The culture medium was then replaced with new medium and treated with 2 μM Flou-4 and 1.5 μM of Rhod-2 (for the detection of mitochondrial calcium) for 30 minutes at 37° C. in the dark. Rhod-2 is a cationic rhodamine-based indicator probe and it can accumulate preferentially in potential-driven uptake in mitochondria. The resting mitochondrial calcium concentration is generally in the range of 100-150 nM. At this level of calcium, the brownish red fluorescence of Rhod-2 is minimal. However, when large excess of calcium ions are uptaken by the mitochondria, concentration-dependent increases in the intensities of Rhod-2 fluorescence will occur. The phenomenon is a testimony of mitochondrial calcium overload.

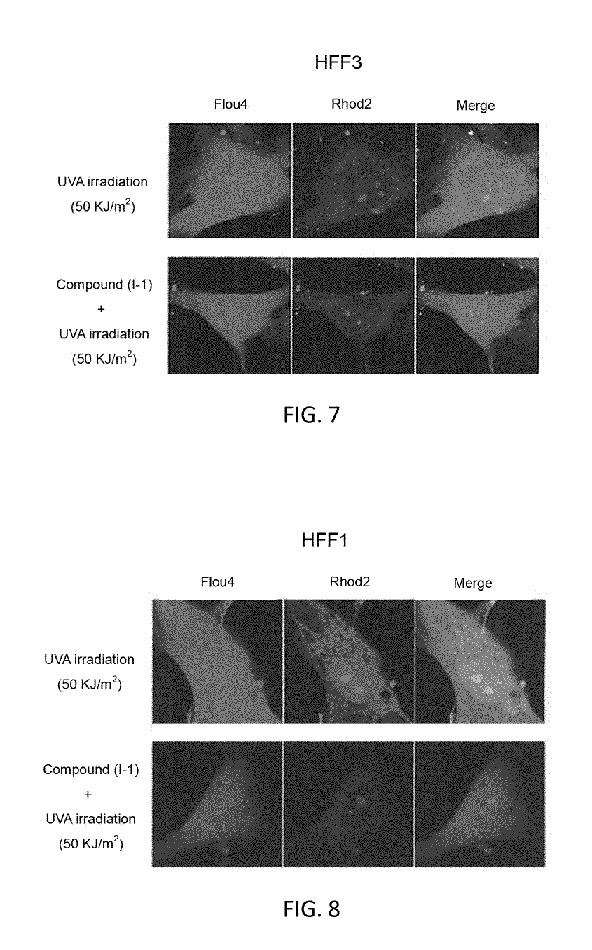

At indicated in FIG. 7 and FIG. 8, when both HFF-3 and HFF-1 cells were irradiated with UVA (50KJ/cm²), an apparent phenomenon of mitochondrial calcium overload had occurred as reflected by the intensity of brownish red fluorescence of Rhod-2 probe. Interestingly, the extent of mitochondrial calcium overload found in HFF-1 cells was much more severe than HFF-3 cells. Remarkably, we found that compound (I-1) (200 μM) could effectively protect HFF-1 cells from UVA-instigated mitochondrial calcium overload as reflected by a drastic decrease in brownish red fluorescence intensity (FIG. 8). Therefore, it can be concluded that compound (I-1) can prevent calcium overload in mitochondria of HFF3 and HFF1 and may further prevent cell apoptosis induced by calcium overload.

[TUNEL Analysis]

Apoptotic cell death was assayed by using an Apo-BrdU in situ DNA fragmentation assay kit. This kit measures the fragmented DNA of apoptotic cells by catalytically incorporating fluorescein-12-dUTP at 3′-OH DNA ends using the terminal deoxynucleotidyl transferase enzyme. The fluorescein-12-dUTP-labeled DNA can then be visualized by confocal scanning microscope and quantified by using software.

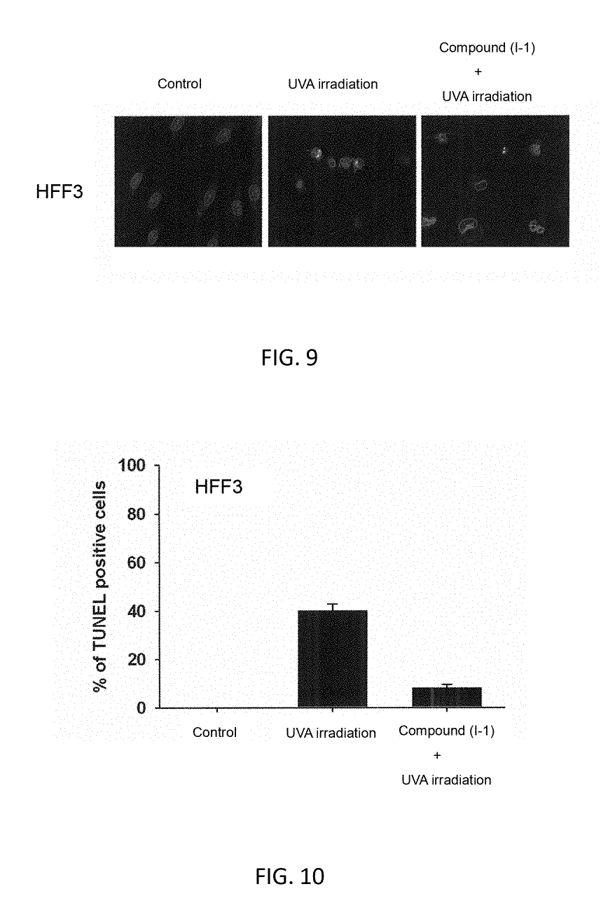

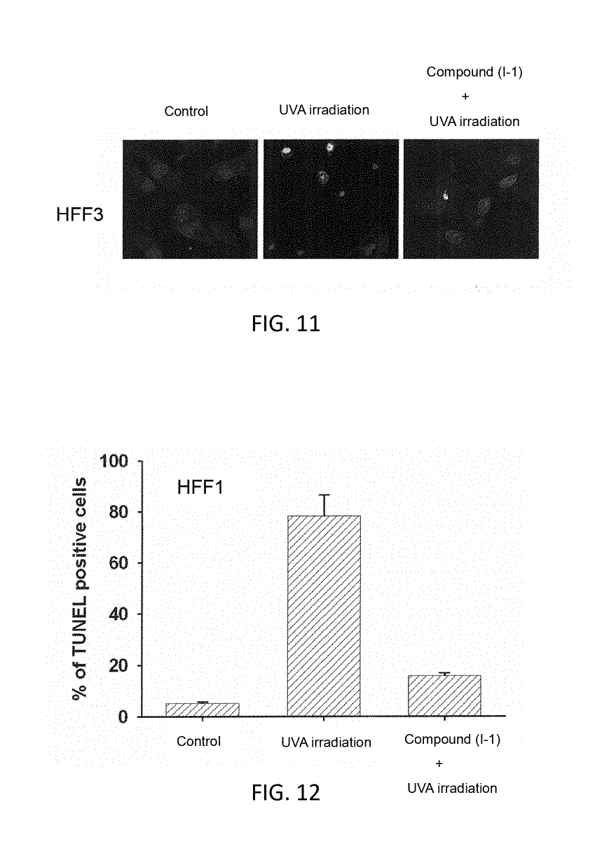

As indicated in FIG. 9 to FIG. 12, when HFF-3 and HFF-1 cells were exposed to UVA (50 KJ/cm²), drastically increased in the numbers of TUNEL-positive population were observed (ca 40% and 80% in HFF-3 and HFF-1, respectively). However, the addition of compound (I-1) (200 μM) prior to UVA exposure, the numbers of TUNEL-positive cell population in HFF-3 and HFF-1 cells significantly dropped to 8% and 18%, respectively. These data imply that compound (I-1) could protect HFF-1 (G6PD-deficient fibroblasts) even more efficiently against UVA-induced apoptotic lethality than HFF-3 (normal fibroblasts) cells. For this reason, it can be speculated that compound (I-1) can mitigate the loss of collagen caused by the apoptosis of fibroblasts instigated by UVA exposure.

According to the aforementioned experimental data, the present invention proves that compound (I) can effectively reduce the intracellular ROS, suppress lipid peroxidation, minify intracellular GSH depletion, prevent calcium (Ca²⁺) overload in mitochondria, and prevent down-regulation of collagen expression in normal fibroblast or G6PD-deficient fibroblast caused by the oxidative damages of UVA.

The skin protection composition of the present invention is specifically designed for G6PD-deficient patients to combat UVA-triggered apoptotic lethality of fibroblasts so that the collagen synthesis and the protection of the structural integrity of collagen can be properly maintained. Our experimental data revealed that when HFF-1 (G6PD-deficient) fibroblasts were exposed to various dosages of UVA irradiation, the production of ROS, as reflected by green DCF fluorescence, was drastically more abundant than their HFF-3 counterparts (normal fibroblasts) due to the blockade of the regeneration of oxidized GSSG back to GSH via GSH reductase because of the deficiency of NADPH cofactor. However, when compound (I-1) (200 μM) was added prior to UVA exposure, a nearly complete suppression of ROS production in HFF-1 cells could be observed which was comparable to the efficacy of HFF-3 cells. This finding clearly substantiated that indeed compound (I-1) is a superb constituent suitable for use to protect the harmful oxidative damage instigated by UVA. In addition, owing to the dramatic rescue of HFF-1 fibroblasts from apoptosis caused by UVA irradiation (50 KJ/cm2) as reflected by drop of the percentage of TUNEL-positive cells from 80% to 18%, the availability of fibroblasts needed for the biosynthesis of collagen induced by UVA-induced ROS insults will result in the overall increased abundance of collagen. This finding implies that compound (I-1) is also an excellent anti-wrinkling ingredient for G6PD-deficient patients.

Our experimental data also revealed that when both HFF-3 and HFF-1 fibroblasts were exposed to UVA irradiation, three apoptosis-related intracellular events could be observed. First, we demonstrated that UVA-triggered lipid peroxidation, as reflected by the fluorescence of probe, was much more severely in HFF-1 cells as compared to HFF-3 cells. However, pretreatment of compound (I-1) prior to UVA irradiation, resulted in the significant decline of green fluorescence of probe by both cell types indicating that UVA-induced lipid peroxidation phenomenon can be blocked by compound (I-1). Second, we substantiated that when both HFF-3 and HFF-1 fibroblasts were exposed to various dosages of UVA irradiation, a progressive decline of the green fluorescence generated by CMF-GSH was observed indicating that the extents of GSH depletion phenomena had occurred. The degrees of GSH depletion were much more severe in HFF-1 cells than HFF-3 cells. Remarkably, pretreatment of compound (I-1) prior to UVA irradiation, a nearly complete recovery of green fluorescence of CMF-GSH complex could be observed. This finding implies that GSH depletion phenomenon is almost completely reversed. Thus, compound (I-1) is a superb agent to prevent UVA-induced GSH depletion. Third, we also demonstrated that when both HFF-3 and HFF-1 fibroblasts were exposed to UVA irradiation (50 KJ/cm2), the mitochondrial calcium overload, as reflected by the brownish red fluorescence of Rhod-2 (an indication of calcium concentration over the threshold level of mitochondrial calcium concentration) significantly increased with HFF-1 cells showing much higher intensity than HFF-3 cells. However, we found that when compound (I-1) was added prior to UVA exposure, the brownish red fluorescence of Rhod-2 could effectively be suppressed indicating a greater recovery of mitochondrial calcium overload has ensued. This finding implied that compound (I-1) is also an effective agent in protecting both cell types from UVA-induced mitochondrial calcium overload. Taken together, the anti-apoptotic effect instigated by compound (I-1) is ascribable to the capability of this agent to suppress UVA-induced lipid peroxidation, GSH depletion and mitochondrial calcium overload.

What is claimed is:

1. A method of protecting skin of glucose-6-phosphate dehydrogenase deficiency patients from ultraviolet radiation, comprising the step of: administering a skin protective composition including an effective amount of a compound (I); 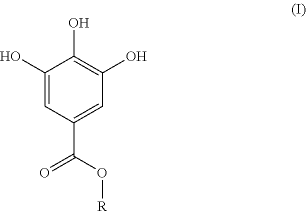
wherein R is unsubstituted C₁₋₆ alkyl group.

2. The method as claimed in claim 1, wherein the skin protective composition is used to prevent ultraviolet A radiation damages.

3. The method as claimed in claim 1, wherein the skin protective composition is used to protect fibroblasts.

4. The method as claimed in claim 1, wherein the skin protective composition is used to reduce ultraviolet-provoked mitochondria calcium (Ca²⁺) overload, ultraviolet-provoked collagen breakdown, ultraviolet-provoked intracellular glutathione depletion, or combination thereof.

5. The method as claimed in claim 1, wherein R is methyl group.

6. A method of protecting skin of a human by reducing ultraviolet-provoked calcium (Ca²⁺) overloading in mitochondria, ultraviolet-provoked collagen breakdown, or combination thereof, comprising the step of: administering a skin protective composition including an effective amount of a compound (I); 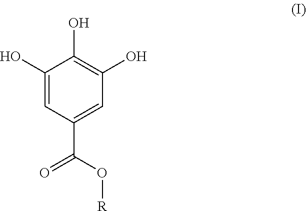
wherein R is unsubstituted C₁₋₆ alkyl group.

7. The method as claimed in claim 6, wherein the skin protective composition is used to prevent ultraviolet A radiation damages.

8. The method as claimed in claim 6, wherein R is methyl group.

9. A method of protecting skin of a human by reducing the adverse effect of ultraviolet radiation on fibroblast, comprising the step of: administering a skin protective composition including an effective amount of a compound (I); 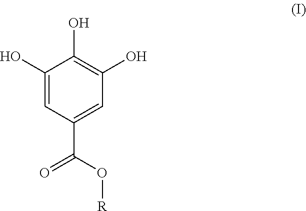
wherein R is methyl group.

10. The method as claimed in claim 9, wherein the skin protective composition is used to reduce ultraviolet-provoked calcium (Ca²⁺) overloading in mitochondria, ultraviolet-provoked collagen breakdown, ultraviolet-provoked intracellular glutathione depletion, or combination thereof.

11. The method as claimed in claim 9, wherein the skin protective composition is used to prevent ultraviolet A radiation damages.
